(19) United States
(12) Patent Application Publication
(10) Pub. No.: US 2024/0082445 A1
(43) Pub. Date: Mar. 14, 2024

(54) Devices and Methods for Detecting the Presence of Nitric Oxide

(71) Applicant: Sterile State LLC, Farmington Hills, MI (US)

(72) Inventors: Megan Cecelia Frost, Trenary, MI (US); Jeffrey Millin, Trenary, MI (US)

(73) Assignee: Sterile State LLC, Farmington Hills, MI (US)

(21) Appl. No.: 18/272,541

(22) PCT Filed: Jan. 26, 2022
(86) PCT No.: PCT/US22/13903

§ 371 Date: Jul. 14, 2023

Related U.S. Application Data
(60) Provisional application No. 63/156,917, filed on Mar. 4, 2021.
(60) Provisional application No. 63/141,676, filed on Jan. 26, 2021.
(60) Provisional application No. 63/141,711, filed on Jan. 26, 2021.

(51) Int. Cl. A61L 2/28; A61L 2/20; G01N 21/78

(52) U.S. Cl. CPC A61L 2/28; A61L 2/20; G01N 21/783

(57) ABSTRACT

A sterilization indicator for detecting the presence, along with quantifying the amount, of nitric oxide is provided herein. The sterilization indicator comprises a chromophore-containing compound capable of undergoing a color change in the presence of nitric oxide. The sterilization indicator further comprises a support layer comprising the chromophore-containing compound. In some embodiments, the sterilization indicator further comprises a polymer layer overlying at least a portion of the support layer for reducing diffusion of a fluid through the polymer layer to the chromophore-containing compound.

### CROSS-REFERENCE TO RELATED APPLICATIONS

This application is an International Application which claims priority to Provisional Patent Application No. 63/141,711, filed on Jan. 26, 2021, Provisional Patent Application No. 63/141,676, filed on Jan. 26, 2021, and Provisional Patent Application No. 63/156,917, filed on Mar. 4, 2021, the entire contents of which are incorporated by reference in their entirety.

### TECHNICAL FIELD

The present disclosure generally relates to sterilization indicators for detecting the presence of nitric oxide, along with quantifying the amount of the same. The present disclosure also relates to methods of forming the sterilization indicators and methods for detecting the presence of nitric oxide.

### BACKGROUND

A variety of products and articles, including, for example, medical instruments, devices, and equipment, must be sterilized prior to use to prevent bio-contamination of a wound site, a sample, an organism, or the like. A number of sterilization processes are used which involve contacting the product or article with a sterilant. Examples of such sterilants include steam, nitric oxide, ethylene oxide, hydrogen peroxide, dry heat, and the like.

Conventional visual indicators are available for steam, hydrogen peroxide, and dry heat. However, suitable visual indicators for nitric oxide are not available. One insufficient indicator for nitric oxide detect nitrites using the Griess Assay rather than detecting for nitric oxide. The Griess Assay does not detect gas phase NO and requires multiple reagents to come together for the reaction to occur. Another insufficient indicator for nitric oxide rely on colorimetric assays that detect nitric assays with either visible spectroscopy or fluorometry that are completed in the solution phase. These reactions require complex gas phase/solution phase equilibria to be established and instrumentation (spectrometers) to determine a level of NO present. This is not conducive with a clinical sterilization need that should give a rapid yes/no indication of sterilization.

Accordingly, it is desirable to provide sterilization indicators detecting the presence of nitric oxide, along with quantifying the amount of the same, and methods for forming and using the sterilization indicators. Furthermore, other desirable features and characteristics will become apparent from the subsequent summary and detailed description and the appended claims, taken in conjunction with the foregoing technical field and background.

### BRIEF SUMMARY

A sterilization indicator for detecting the presence of nitric oxide is provided herein. The sterilization indicator comprises a chromophore-containing compound capable of undergoing a color change in the presence of nitric oxide. The sterilization indicator further comprises a support layer comprising the chromophore-containing compound. In some embodiments, the sterilization indicator further comprises a polymer layer overlying at least a portion of the support layer for reducing diffusion of a fluid through the polymer layer to the chromophore-containing compound.

A sterilization indicator for quantifying the amount of nitric oxide is also provided herein. The sterilization indicator comprises a chromophore-containing compound capable of undergoing a color change in the presence of nitric oxide. The sterilization indicator further comprises a support layer comprising the chromophore-containing compound. In some embodiments, the sterilization indicator further comprises a polymer layer overlying at least a portion of the support layer for reducing diffusion of a fluid through the polymer layer to the chromophore-containing compound.

A method of detecting the presence of nitric oxide within a space is also provided herein. The method comprises providing a nitric oxide source for exposing the space to nitric oxide. The method further comprises providing a sterilization indicator to the space. The method further comprises exposing the sterilization indicator to the nitric oxide. The method further comprises observing a color change of the sterilization indicator after exposure of the sterilization indicator to the nitric oxide for a predetermined period of time to detect the presence of nitric oxide within the space.

A method of quantifying the amount of nitric oxide within a space is also provided herein. The method comprises providing a nitric oxide source for exposing the space to nitric oxide. The method further comprises providing a sterilization indicator to the space. The method further comprises exposing the sterilization indicator to the nitric oxide. The method further comprises observing a color change of the sterilization indicator after exposure of the sterilization indicator to the nitric oxide for a predetermined period of time to quantifying the amount of nitric oxide within the space.

A method of forming a sterilization indicator for detecting the presence and amount of nitric oxide is also provided herein. The method comprises providing a support layer. The method further comprises providing a chromophore-containing compound capable of undergoing a color change in the presence of nitric oxide. The method further comprises combining the support layer and the chromophore-containing compound. In some embodiments, the method further comprises applying a polymer layer to at least a portion of the support layer to form the sterilization indicator.

A method of forming a sterilization indicator for quantifying the amount of nitric oxide is also provided herein. The method comprises providing a support layer. The method further comprises providing a chromophore-containing compound capable of undergoing a color change in the presence of nitric oxide. The method further comprises combining the support layer and the chromophore-containing compound. In some embodiments, the method further comprises applying a polymer layer to at least a portion of the support layer to form the sterilization indicator.

In non-limiting embodiments, a solid-state sterilization indicator that allows a visible dye or chromophore to change color from clear to bright green upon exposure to gas phase nitric oxide is provided. However, it is to be appreciated that other dyes can be used resulting in various color transitions (e.g. yellow to red). The chromophore may be infused in a solid matrix, such a cellulose, and top coated on all sides with a polymer, such as polyvinyl chloride (PVC) to control diffusion of oxygen and nitric oxide to the dye or chromophore. Cellulosed-based tape (e.g., cellophane) and polypropylene tape may also be utilized as diffusion barriers to control contact of the nitric oxide to the dye or chromophore. The amount of dye or chromophore and the identity and thickness of the top coating/diffusion layer can be adjusted to tune the sensitivity and timing of response of the sterilization indicator to the nitric oxide.

### BRIEF DESCRIPTION OF THE DRAWING(S)

FIG. 1 is a cross-sectional perspective view illustrating a non-limiting embodiment of a sterilization indicator.

FIG. 2 is a cross-sectional perspective view illustrating another non-limiting embodiment of the sterilization indicator.

FIG. 3 is a cross-sectional perspective view illustrating another non-limiting embodiment of the sterilization indicator.

FIG. 4 is a photograph of various time points of a comparative sterilization indicator.

FIG. 5 is a photograph of various time points of a non-limiting embodiment of an exemplary sterilization indicator.

FIG. 6 is a graph illustrating color change over time for a non-limiting embodiment of an exemplary sterilization indicator.

FIG. 7 is a photograph of various non-limiting embodiments of an exemplary sterilization indicator.

FIG. 8 is a perspective view illustrating a non-limiting embodiment of an article to be sterilized or sanitized.

FIG. 9 is a perspective view illustrating a non-limiting embodiment of a device including the sterilization indicator of FIGS. 1-3.

FIG. 10 is a cross-sectional perspective view illustrating a non-limiting embodiment of a support of the device of FIG. 9.

FIG. 11 is a cross-sectional perspective view illustrating a non-limiting embodiment of a case for the support of FIG. 10.

FIG. 12 is a photograph of a non-limiting embodiment of a barrier including the sterilization indicator of FIGS. 1-3.

### DETAILED DESCRIPTION

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g. a total weight of dry matter).

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed so as to have the same overall result as if the object were completely enclosed.

The drawings are semi-diagrammatic and not to scale and, particularly, some of the dimensions are for the clarity of presentation and are shown exaggerated in the drawings. Similarly, although the views in the drawings for ease of description generally show similar orientations, this depiction in the drawings is arbitrary. Generally, the sterilization indicator can be operated in any orientation. As used herein, it will be understood that when a first element or layer is referred to as being “over,” “overlying,” “under,” or “underlying” a second element or layer, the first element or layer may be directly on the second element or layer, or intervening elements or layers may be present where a straight line can be drawn through and between features in overlying relationship. When a first element or layer is referred to as being “on” a second element or layer, the first element or layer is directly on and in contact with the second element or layer. Further, spatially relative terms, such as “upper,” “over,” “lower,” “under,” and the like, may be used herein for ease of description to describe one element or feature's relationship to another element(s) or feature(s) as illustrated in the figures. It will be understood that the spatially relative terms are intended to encompass different orientations of the sterilization indicator in use or operation in addition to the orientation depicted in the figures. For example, if the sterilization indicator in the figures is turned over, elements described as being “under” other elements or features would then be oriented “above” the other elements or features. Thus, the exemplary term “under” can encompass either an orientation of above or below. The sterilization indicator may be otherwise oriented (rotated 90 degrees or at other orientations) and the spatially relative descriptors used herein may likewise be interpreted accordingly.

Throughout this disclosure, where publications are referenced, the disclosures of these publications in their entireties are hereby incorporated by reference into this disclosure to more fully describe the state of the art to which this disclosure pertains.

The following detailed description is merely exemplary in nature and is not intended to limit the invention or the application and uses of the invention. Furthermore, there is no intention to be bound by any theory presented in the preceding background or the following detailed description.

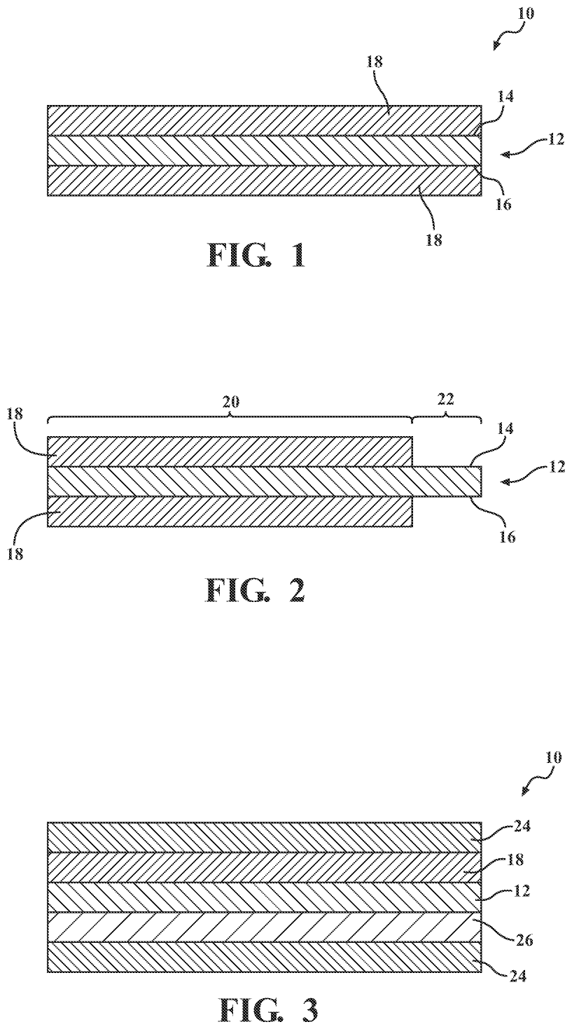

FIG. 1 is a cross-sectional perspective view illustrating a non-limiting embodiment of a sterilization indicator 10 for detecting the presence of nitric oxide. The sterilization indicator 10 comprises a chromophore-containing compound capable of undergoing a color change in the presence of nitric oxide. In various embodiments, the chromophore-containing compound comprises 2,2′-azinobis(3-ethylbenzothiazoline-6-sulfonic acid) (“ABTS”), methyl organge (MeORG), thymolblue (ThBlu), or combinations thereof. It is to be appreciated that any chromophore-containing compound capable of undergoing a color change in the presence of nitric oxide may be utilized. In one exemplary embodiment, the chromophore-containing compound comprises, consists essentially of, or is, ABTS. Without being limited to theory, it is believed that ABTS oxidizes to form its radical cation in the presence of nitric oxide, as shown below:

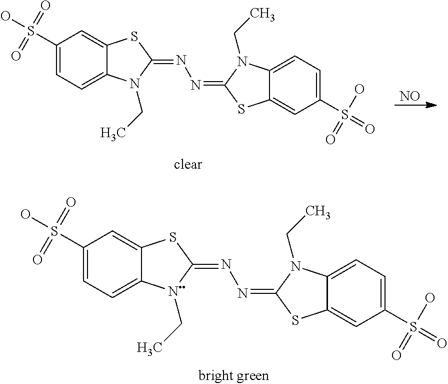

As a result of oxidation in the presence of nitric oxide, the clear ABTS undergoes a color change to form the bright green-colored ABTS radical cation. Therefore, in various embodiments, this oxidation in the presence of nitric oxide results in the sterilization indicator 10 undergoing a color-change (e.g., from clear to bright green-colored). However, it is to be appreciated that the sterilization indicator 10 may undergo any combination of color changes depending on the constituents of the sterilization indicator 10.

The sterilization indicator 10 further comprises a support layer 12. The support layer 12 may have a first surface 14 and a second surface 16 opposite the first surface 14. However, it is to be appreciated that the support layer 12 may have any number of surfaces, such as 3, 4, 5, 6, etc. In various embodiments, the support layer 12 has a rectangular configuration or an ovular configuration. However, it is to be appreciated that the support layer 12 may have any geometrical configuration suitable to support the sterilization indicator 10. The support layer 12 may have a thickness in an amount of from about 0.1 microns to about 1000 microns, optionally from about 1 to about 100, or optionally from about 1 to about 10, mils.

The support layer 12 comprises the chromophore-containing compound. In some embodiments, the support layer 12 has a porous or weave-like structure and the chromophore-containing compound is disposed within the pores or gaps within the weave-like structure. In other embodiments, the chromophore-containing compound may be combined with a moldable material to form the support structure 12 comprising the chromophore-containing compound. In certain embodiments, the support layer 12 comprises, consists essentially of, consists of, or is, a cellulose-containing material. Non-limiting examples of suitable cellulose-containing materials include cellulose filter paper, such as Whatman no. 1 quantitative filter paper.

The chromophore-containing compound may be included dispersed within in or disposed on the support structure 12, or both. The chromophore-containing compound may be evenly dispersed within in or disposed on the support structure 12, or the chromophore-containing compound may be present as a gradient relative to the configuration of the support structure 12. In certain embodiments, the chromophore-containing compound is disposed evening throughout the support structure 12. The chromophore-containing compound may be present within in or disposed on the support structure 12 in an amount of at least trace quantities, optionally at least 6.9 mg/mL, or optionally at least 25 mg/mL, based on a total surface area of the support structure 12.

The sterilization indicator 10 further comprises a polymer layer 18 overlying at least a portion of the support layer 12 for reducing diffusion of a fluid through the polymer layer to the chromophore-containing compound. In various embodiments, the fluid may be a liquid or a gas, such as oxygen, nitric oxide, or hydrogen peroxide. In certain embodiments, the polymer layer 18 may be adapted to reduce diffusion of hydrogen peroxide to a greater extent than the diffusion of nitric oxide to the chromophore-containing compound. In various embodiments, the polymer layer 18 may formed from a polymeric material having a diffusion rate for nitric oxide in an amount of no greater than the diffusion rate of NO in air. [In these and other embodiments, the polymer layer 18 may be formed from a polymeric material having a diffusion rate for oxygen in an amount of no greater than the diffusion rate in air. In these and other embodiments, the polymer layer 18 may formed from a polymeric material having a diffusion rate for hydrogen peroxide in an amount of no greater than the diffusion rate in air.

The polymer layer 18 may be formed from a polymeric material comprising polyvinyl chloride, polyester, a cellulose-containing material, polypropylene, or combinations thereof. However, it is to be appreciated that any other polymeric material may be utilized to for the polymeric layer 18 so long as the polymeric material exhibits the diffusion characteristics described above and the polymeric material is inert relative to the chromophore-containing compound. The polymer layer 18 may have a thickness in an amount of from about 0.1 microns to about 1000 microns, optionally from about 1 to about 100, or optionally from about 5 to about 10 mils.

The polymeric material may further include a variety of additives, including, but not limited to, a solvent component, a plasticizer component, a surfactant component, a colorant component, a filler component, or combinations thereof.

The solvent component may include an organic solvent. However, it is to be appreciated that the solvent component may include any other solvent, including water, known for solvating solutes so long as the solvent is compatible with the components of the polymeric material and the chromophore-containing compound.

Examples of suitable organic solvents for the solvent component include, but are not limited to, toluene, xylene, butyl acetate, acetone, methyl isobutyl ketone, methyl ethyl ketone, ethyl amyl ketone, methanol, isopropanol, butanol, hexane, acetone, ethylene glycol, monoethyl ether, propylene glycol methyl ether, VM and P naptha, mineral spirits, heptane and other aliphatic, cycloaliphatic, aromatic hydrocarbons, aromatic petroleum distillates, esters, ethers and ketone, or combinations thereof. In certain embodiments, the solvent component includes methyl ethyl ketone (MEK), methyl isobutyl ketone (MIBK), toluene, propylene glycol methyl ether, or combinations thereof.

The plasticizer component may include a plasticizer that may be used to modify various characteristics including, but not limited to, coating hardness, adding hydrophobicity, and/or modifying diffusion of fluids, and the like. The plasticizer includes, but is not limited to, phthalates, trimellitates, benzoates, adipates, sebacates, maleates, citrates, epoxidized vegetable oils, sulfonamides, organophosphates, glycols/polyethers, polymeric plasticizers and polybutenes, or combinations thereof. However, it is to be appreciated that the plasticizer component may include any other plasticizer understood in the art so long as the plasticizer is compatible with the components of the polymeric material and the chromophore-containing compound.

The plasticizer may be an ester plasticizer. Examples of suitable ester plasticizers include, but are not limited to, dioctyl phthalate (DOP), n-hexyl-n-decyl phthalate (NHDP), n-octyl-decyl phthalate (NODP), di(isononyl) phthalate (DINP), di(isodecyl)phthalate (DIDP), diundecyl phthalate (DUP), di(isotridecyl)phthalate (DTDP), di-2-ethylhexyl adipate (DOA), di-n-octyl-n-decyl adipate (DNODA), diisononyl adipate (DINA), di-2-ethylhexyl azelate (DOZ), di-2-ethylhexyl sebacate (DOS), trioctyl trimellitate (TOTM), trioctyl phosphate (TOP), tricresyl phosphate (TCP), aliphatic polyester plasticizer, aliphatic polyol plasticizer, or combinations thereof. It is to be appreciated the plasticizer may include any phthalate known in the art so long as it is compatible with the components of the polymeric material and the chromophore-containing compound. The plasticizer component may be present in the polymeric material in various amounts.

The surfactant component may include anionic surfactants, non-ionic surfactants, cationic surfactants, Zwitterionic surfactants, or combinations thereof. However, it is to be appreciated that the surfactant component may include any other surfactant understood in the art so long as the surfactant is compatible with the components of the polymeric material and the chromophore-containing compound. The surfactant component may be present in the polymeric material in various amounts.

The colorant component may include a colorant including, but not limited to, one or more pigments, dyes, or combinations thereof to achieve a coating color. These colorants are in addition to the chromophore-containing compound. Suitable colorants will generally be those that are soluble or dispersible in the solvent component of the polymeric material and the chromophore-containing compound. The colorant component may be present in the polymeric material in various amounts.

The filler component may include a filler that may be used for various objectives including, but not limited to, cost control, rheology control, lubricity modification, as well as to prevent seizing or galling. The filler component may include an inorganic filler. Examples of suitable inorganic fillers include, but are not limited to, powdered nickel, copper, zinc, and aluminum. Suitable mineral fillers include, but are not limited to, talc, calcium carbonate, silicates such as mica, wollastonite, titanium dioxide, quarts, fumed silica precipitated silica, graphite, boron nitride, or combinations thereof. The filler component may be present in the polymeric material in various amounts.

Beyond the components of the polymer layer 18 described above, there are several factors that can impact diffusion of the fluids to the chromophore-containing compound and reactivity of the nitric oxide with the chromophore-containing compound. Non-limiting examples of these factors include temperature of the space, humidity level of the space, acidity of the polymer layer 18, thickness of the polymer layer 18, and concentration of the chromophore-containing compound.

FIG. 2 is a cross-sectional perspective view illustrating another non-limiting embodiment of the sterilization indicator 10. In certain embodiments, the support layer 12 comprises a first portion 20 and a second portion 22 adjacent the first portion 20. In these and other embodiments, the first portion 20 comprises the polymer layer 18 and the second portion 22 is substantially free of the polymer layer 18. By allowing the second portion 22 of the support layer 12 to be directly exposed to the nitric oxide, the sterilization indicator 10 can be adapted to function as a linear timing dosimeter. This linear timing dosimeter provides progressive migration of the nitric oxide from the second portion 22 to and through the first portion 20 with the migration distance being linearly proportional to time of exposure to nitric oxide for a predetermined period of time. In some embodiments, the linear timing dosimeter was linearly proportional for at least 2, optionally at least 3 hours, or optionally at least 4 hours.

FIG. 3 is a cross-sectional perspective view illustrating another non-limiting embodiment of the sterilization indicator 10. The polymer layer 18 may be overlying at least a portion of the first surface 14 of the support layer 12 while the second surface 16 of the support layer 12 remains substantially free of the polymer layer 18. However, as described above, the polymer layer 18 may be overlying at least a portion of the second surface 16 as well.

In various embodiments, the sterilization indicator 10 further includes a backing layer 24 overlying the first surface 14. The backing layer 24 can be in a variety of forms including, e.g., polymer films, paper, cardboard, stock card, woven and nonwoven webs, fiber reinforced films, foams, composite film-foams, or combinations thereof. The backing layer 24 can include a variety of materials including, e.g., fibers, lignocellulose, wood, foam, and thermoplastic polymers including, e.g., polyolefins (e.g., polyethylene including, e.g., high density polyethylene, low density polyethylene, linear low density polyethylene, and linear ultra-low density polyethylene), polypropylene, and polybutylenes; vinyl copolymers (e.g., polyvinyl chlorides, plasticized and un-plasticized polyvinyl chlorides, and polyvinyl acetates); olefinic copolymers including, e.g., ethylene/methacrylate copolymers, ethylene/vinyl acetate copolymers, acrylonitrile-butadiene-styrene copolymers, and ethylene/propylene copolymers; acrylic polymers and copolymers; polyurethanes; and combinations thereof. Suitable blends also include, e.g., blends of thermoplastic polymers, elastomeric polymers and combinations thereof including, e.g., polypropylene/polyethylene, polyurethane/polyolefin, polyurethane/polycarbonate, and polyurethane/polyester.

In these and other embodiments, the sterilization indicator 10 further comprises an adhesive layer 26 overlying the second surface 16. The adhesive layer 26 can be based on a variety of adhesives. Non-limiting examples of suitable adhesives include various pressure sensitive adhesives, such as water-insoluble natural rubber-based adhesives, natural rubber and synthetic rubber blend adhesives, styrene-isoprene-styrene block copolymers with tackifying resins, vinyl ethers, and high molecular weight acrylate copolymers. Various water-dispersible, pressure sensitive adhesives may also be utilized. It is to be appreciated that another backing layer 24 may overly the adhesive layer 26.

A method of detecting the presence of nitric oxide within a space is also provided. The method comprises providing a nitric oxide source for exposing the space to nitric oxide. The method further comprises providing the sterilization indicator 10 to the space. The method further comprises exposing the sterilization indicator 10 to the nitric oxide. The method further comprises observing a color change of the sterilization indicator 10 after exposure of the sterilization indicator 10 to the nitric oxide for a predetermined period of time to identify the presence of nitric oxide within the space.

In some embodiments, the space and objects within the space are desired to be sanitized or sterilized using nitric oxide. Non-limiting examples of suitable spaces include medical examination rooms, classrooms, restaurants, aircraft cabins, vehicle interiors, etc. In these embodiments, the method further comprises discontinuing exposure of the space to nitric oxide after a color change of the sterilization indicator 10 has been observed.

In other embodiments, the space may be a container or receptacle including an article desired to be sanitized and then removed from the space. Non-limiting examples of suitable articles include medical equipment, educational materials, handheld devices, food service equipment, etc. In these embodiments, the method further comprises providing the article to be sterilized to the space, exposing the article to the nitric oxide, and removing the article from the space.

In certain embodiments, the space is disposed within an environment and the space is substantially fluidly-isolated from the environment. The phrase “substantially fluidly-isolated” means that the movement of fluid into the space from the environment, and vice versa, is minimized. However, it is to be appreciated that the space does not need to be hermetically isolated from the environment (although it can be) for the sterilization indicator 10 to be operable.

Use of the sterilization indicator 10 to identify the presence of nitric oxide within a space is also provided, in accordance with the foregoing.

A method of forming a sterilization indicator 10 for detecting the presence of nitric oxide is also provided. The method comprises providing the support layer 12. The method further comprises providing the chromophore-containing compound capable of undergoing a color change in the presence of nitric oxide. The method further comprises combining the support layer 12 and the chromophore-containing compound. The method further comprises applying the polymer layer 18 to at least a portion of the support layer 12 to form the sterilization indicator 10.

In various embodiments, the method further comprises drying the support layer 12 combined with the chromophore-containing compound in the presence of nitrogen prior to applying the polymer layer 18.

In certain embodiments, as described above with regard to the linear timing dosimeter embodiment, the step of applying a polymer layer 18 to at least a portion of the support layer 12 is further defined as applying the polymer layer 18 to the first portion 20 of the support layer 12 such that the second portion 22 of the support layer 12 is substantially free of the polymer layer 18.

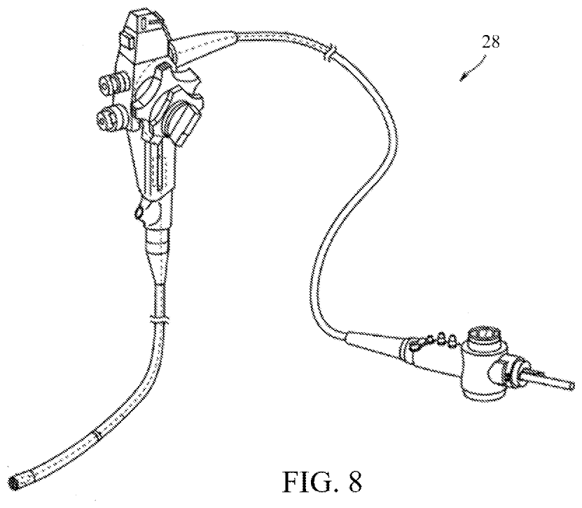

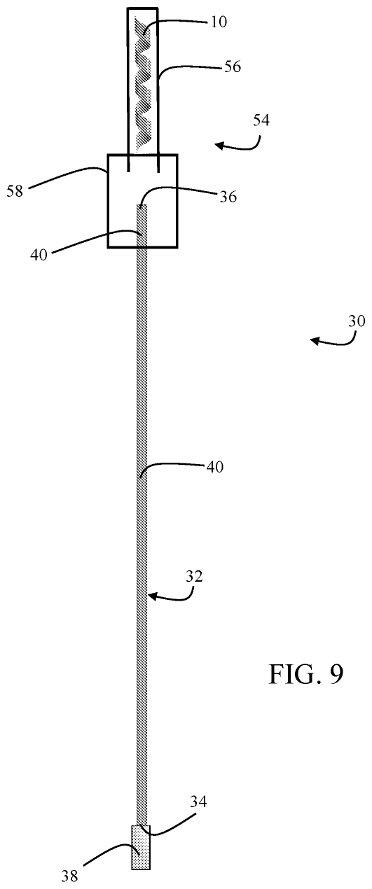

FIGS. 8-12 are various views of components of a system including the sterilization indicator 10 for detecting the presence of nitric oxide proximate an article 28. With reference to FIGS. 8 and 9, the system may include the article 28 defining a void, such as a lumen of an endoscope. The system may further include a device 30 for providing nitric oxide for sanitizing or sterilizing the article 28, such as an optical fiber. Non-limiting examples of suitable sterilization techniques are described in U.S. Pat. App. Nos. 63/141,676 and 63/156,917, which are incorporated by reference in their entirety. The device 30 may include a support 32 having a surface with the surface adapted to transmit electromagnetic radiation. The support 32 may have a first end 34 and a second end 36 spaced from the first end 34.

The device 30 may further include an electromagnetic radiation source 38 in optical communication with the support 32 and adapted to generate electromagnetic radiation. In certain embodiments, the electromagnetic radiation source 38 includes an LED bulb that is coupled to the first end 34.

With reference to FIG. 9, the device 30 may further include a nitric oxide source 40 disposed on the surface of the support 32. Non-limiting examples of suitable nitric oxide sources are described in U.S. Pat. App. Nos. 63/141,676 and 63/156,917, which are incorporated by reference in their entirety. In certain embodiments, the nitric oxide source 40 includes SNAP-PDMS or other nitric oxide sources. To this end, the nitric oxide source 40 is adapted to provide nitric acid in the presence of the electromagnetic radiation generated by the electromagnetic radiation source 38 and transmitted through the support 32 from the first end 34 to the second end 36. The support 32 of the device 30 may be disposed within the lumen of the article 28 (e.g., an endoscope).

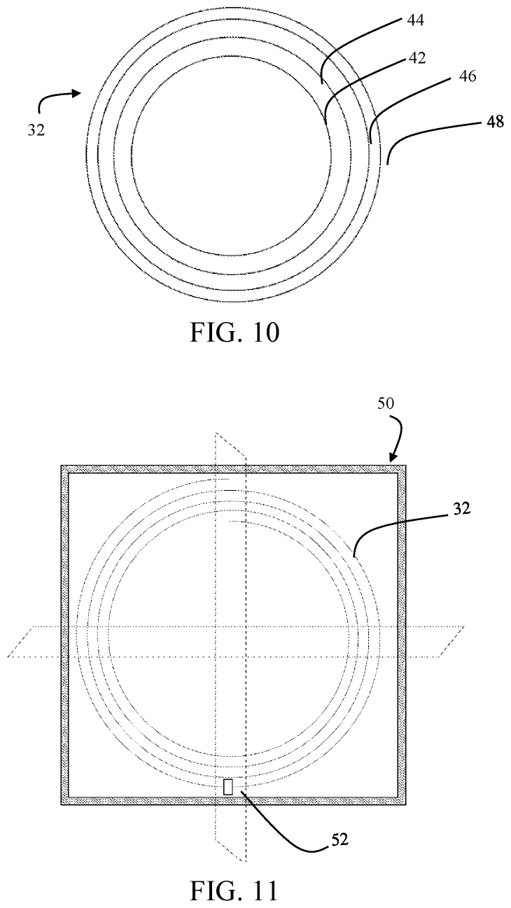

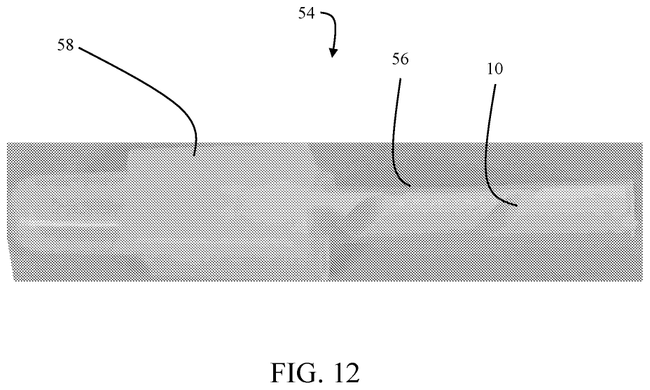

In various embodiments, with reference to FIGS. 10 and 11, the support 32 is an optical fiber including a PMMA core 42, a cladding layer 44, the nitric oxide source 46, and a PDMS protective layer 48. The support 32 may be wound in a case 50 formed from foam to permit insertion of the support 32 into the article 28 without contaminating the support 32. The case 50 may include a cap 52 for accessing the support 32.

Referring to FIGS. 9 and 12, the device 30 may further include the sterilization indicator 10 proximate the second end 36 of the support 32. The sterilization indicator 10 may be disposed within a barrier 54 that is coupled to the second end 36 of the support 32. In certain embodiment, the barrier 54 includes an indicator portion 56 and a locking portion 58 with the locking portion 58 coupling the indicator portion 56 to the second end 36 of the support 32. The sterilization indicator 10 may be fluidly isolated from the exterior of the barrier 54. This isolation of the sterilization indicator 10 provides a direct indication of the presence of nitric oxide formed from the support 32 of the device 30 without interference by any nitric oxide sources outside the barrier 54 thereby providing a user feedback regarding the generation of the nitric oxide by the device 30.

Although the invention has been described in connection with specific preferred embodiments, it should be understood that the invention as claimed should not be unduly limited to these specific embodiments. While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the disclosure as set forth in the appended claims.

### INDUSTRIAL APPLICABILITY

While the present invention is not limited to a particular end application, use or industry, hospitals, schools, restaurants, airlines, and public transit operators often rely on sanitation or sterilization. The sterilization indicator is useful for detecting the presence of nitric oxide for sanitation or sterilization.

The following examples, illustrating the sterilization indicator of this disclosure, are intended to illustrate and not to limit the invention.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1 (Comparative)

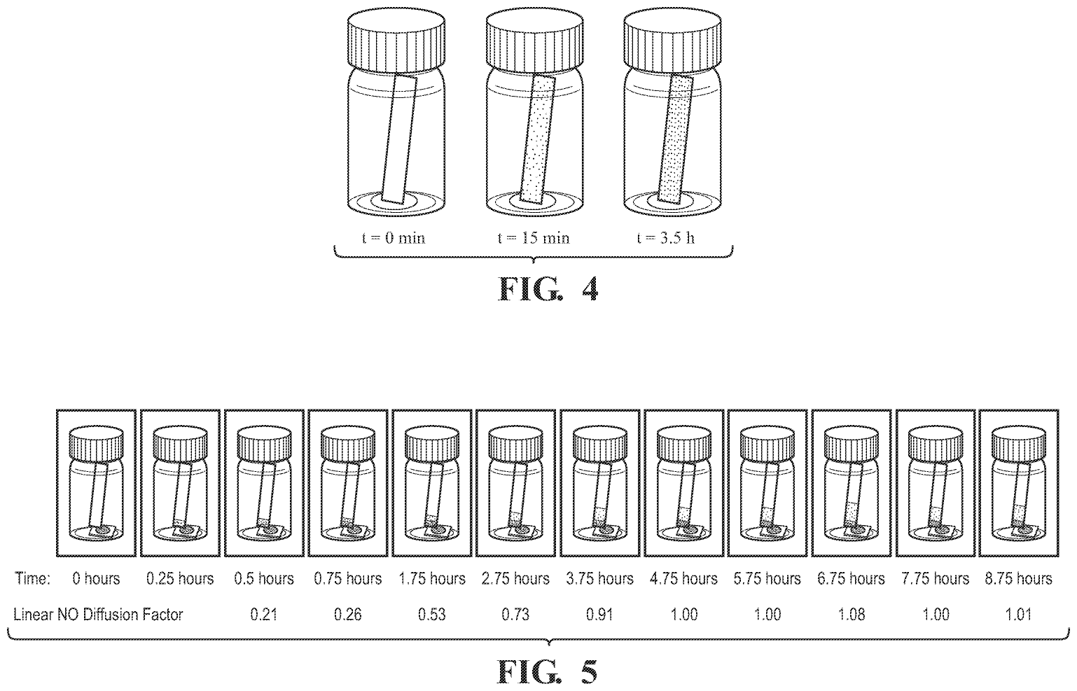

With reference to FIG. 4, cellulose filter paper (Whatman no. 1 quantitative filter paper) was dipped into an aqueous solution of 8.4 mg/mL of 2,2′-Azinobis(3-ethylbenzthiazoline-6-sulfonic acid) (ABTS) and dried under nitrogen to form Comparative Sterilization Indicator I. The Comparative Sterilization Indicator I was then exposed to NO gas and after approximately 15 min began turning dark green. Comparative Sterilization Indicator I reached the final color change after approximately 3.5 hours

### Example 2 (Comparative)

Cellulose filter paper (Whatman no. 1 quantitative filter paper) was saturated with a methanol solution of 6.9 mg/mL of 2,2′-Azinobis(3-ethylbenzthiazoline-6-sulfonic acid) (ABTS) and allowed to dry under nitrogen to form Comparative Sterilization Indicator II. Comparative Sterilization Indicator II was then exposed to NO gas and after approximately 3.5 minutes, Comparative Sterilization Indicator II turned light green. Sterilization Indicator II turned dark green after approximately 24 hours.

### Example 3 (Exemplary)

Cellulose filter paper (Whatman no. 1 quantitative filter paper) was dipped into an aqueous solution of 8.4 mg/mL of 2,2′-Azinobis(3-ethylbenzthiazoline-6-sulfonic acid) (ABTS) and dried under nitrogen to form Exemplary Sterilization Indicator I. Exemplary Sterilization Indicator I was then coated with 5 wt % PVC dissolved in THF. Exemplary Sterilization Indicator I was then exposed to NO gas and after approximately 15 minutes turned green and the intensity of color under the top coat was less than Comparative Sterilization Indicators I and II above.

### Example 4 (Exemplary)

Cellulose filter paper (Whatman no. 1 quantitative filter paper) was saturated with a methanol solution of 6.9 mg/mL of 2,2′-Azinobis(3-ethylbenzthiazoline-6-sulfonic acid) (ABTS) and allowed to dry under nitrogen. The paper was then coated with 5 wt % PVC dissolved in THF to form Exemplary Sterilization Indicator II. Exemplary Sterilization Indicator II was then exposed to NO gas and after approximately 15 minutes turned dark green, the intensity of color under the top coat was less than Comparative Sterilization Indicators I and II above.

### Example 5 (Exemplary)

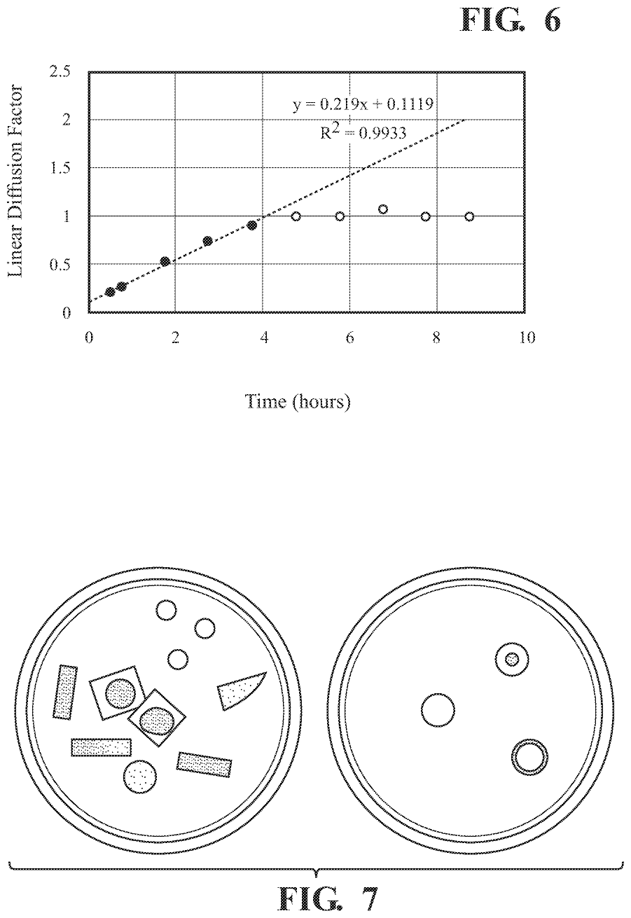

With references to FIG. 5, a linear, passive timer was fabricated by dipping a strip of the cellulose filter paper (Whatman no. 1 quantitative filter paper) into an aqueous solution of 8.4 mg/mL of 2,2′-Azinobis(3-ethylbenzthiazoline-6-sulfonic acid) (ABTS) and drying under nitrogen. Then, the paper was encased all but about 4 mm of the lower end in cellophane tape to form Exemplary Sterilization Indicator III. The lower end strip of Exemplary Sterilization Indicator III was then exposed to NO gas. The color rapidly developed at the lower, exposed end and then progressively migrated up the portion of the strip encased in tape. Color developed linearly relative to time of exposure and migration distance for the first approximate 4 hours of the exposure for NO, as shown in FIG. 6.

### Example 6 (Exemplary)

With reference to FIG. 7, dot timing dosimeters were also formed using 2 different configurations. In the first configuration, the outer edge of the circle was exposed to allow NO sensing with the center of the circle covered in a diffusion barrier to create an “outward-in” dosimeter that shows progress toward NO exposure as the dot develops color from the outer edge inward. In the second configuration, the center of the dot was exposed with the outer edge including the diffusion barrier. This configuration resulted in color developing in the middle of the dot first and continuing to develop in an “in-outward” manner. These symmetrical dosimeters were not sensitive to the physical orientation relative to the NO source.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A sterilization indicator for detecting the presence of nitric oxide, comprising:
a chromophore-containing compound capable of undergoing a color change in the presence of nitric oxide; and
a support layer comprising the chromophore-containing compound.

2. The sterilization indicator of claim 1, wherein the chromophore-containing compound comprises 2,2′-azinobis(3-ethylbenzothiazoline-6-sulfonic acid), methyl organge (MeORG), thymolblue (ThBlu), or combinations thereof.

3. The sterilization indicator of claim 1, wherein the support layer comprises a cellulose-containing material.

4. The sterilization indicator of claim 1, further comprising a polymer layer overlying at least a portion of the support layer for reducing diffusion of a fluid through the polymer layer to the chromophore-containing compound, wherein the polymer layer is formed from a polymeric material comprising polyvinyl chloride, polyester, a cellulose-containing material, polypropylene, or combinations thereof.

5. The sterilization indicator of claim 4, wherein the polymer layer is formed from a polymeric material having:
(A) a diffusion rate for nitric oxide in an amount of no greater than the diffusion rate of nitric oxide in air;
(B) a diffusion rate for oxygen in an amount of no greater than the diffusion rate of oxygen oxide in air;
(C) a diffusion rate for hydrogen peroxide in an amount of no greater than the diffusion rate of hydrogen in air; or
any combination of (A), (B) and (C).

6. The sterilization indicator of claim 4, wherein the support layer comprises a first portion and a second portion adjacent the first portion, and wherein the first portion comprises the polymer layer and the second portion is substantially free of the polymer layer.

7. The sterilization indicator of claim 4, wherein the support layer has a first surface and a second surface opposite the first surface, and wherein the polymer layer is overlying at least a portion of the first surface.

8. The sterilization indicator of claim 7 further comprises a backing layer overlying the first surface.

9. The sterilization indicator of claim 7 further comprises an adhesive layer overlying the second surface.

10. A method of detecting the presence of nitric oxide within a space, the method comprising:
providing a nitric oxide source for exposing the space to nitric oxide;
providing a sterilization indicator to the space, wherein the sterilization indicator is as set forth in claim 1;
exposing the sterilization indicator to the nitric oxide; and
observing a color change of the sterilization indicator after exposure of the sterilization indicator to the nitric oxide for a predetermined period of time to detect the presence of nitric oxide within the space.

11. The method of claim 10 further comprising:
providing an article to be sterilized to the space;
exposing the article to the nitric oxide; and
removing the article from the space.

12. The method of claim 10 further comprising discontinuing exposure of the space to nitric oxide.

13. The method of claim 10, wherein the space is disposed within an environment and the space is substantially fluidly-isolated from the environment.

14. (canceled)

15. A sterilization indicator for quantifying the amount of nitric oxide, comprising:
a chromophore-containing compound capable of undergoing a color change in the presence of nitric oxide;
a support layer comprising the chromophore-containing compound; and
a polymer layer overlying at least a portion of the support layer for reducing diffusion of a fluid through the polymer layer to the chromophore-containing compound.

16. A method of quantifying the amount of nitric oxide within a space, the method comprising:
providing a nitric oxide source for exposing the space to nitric oxide;
providing a sterilization indicator to the space, wherein the sterilization indicator is as set forth in claim 15;
exposing the sterilization indicator to the nitric oxide; and
observing a color change of the sterilization indicator after exposure of the sterilization indicator to the nitric oxide for a predetermined period of time to quantify the amount of nitric oxide within the space.

17. (canceled)

18. A method of forming a sterilization indicator for detecting the presence of nitric oxide, the method comprising:
providing a support layer;
providing a chromophore-containing compound capable of undergoing a color change in the presence of nitric oxide; and
combining the support layer and the chromophore-containing compound to form the sterilization indicator.

19. The method of claim 18 further comprising applying a polymer layer to at least a portion of the support layer to form the sterilization indicator.

20. The method of claim 19 further comprising drying the support layer combined with the chromophore-containing compound in the presence of nitrogen prior to applying the polymer layer.

21. The method of claim 19, wherein the support layer comprises a first portion and a second portion adjacent the first portion, and wherein the step of applying a polymer layer to at least a portion of the support layer is further defined as applying the polymer layer to the first portion such that the second portion is substantially free of the polymer layer.

22. A method of forming a sterilization indicator for quantifying the amount of nitric oxide, the method comprising:
providing a support layer;
providing a chromophore-containing compound capable of undergoing a color change in the presence of nitric oxide;
combining the support layer and the chromophore-containing compound; and
applying a polymer layer to at least a portion of the support layer to form the sterilization indicator.
